(19) United States
(12) Patent Application Publication
(10) Pub. No.: US 2023/0005603 A1
(43) Pub. Date: Jan. 5, 2023

(54) INTELLIGENT EMERGENCY TRIAGE SYSTEM

(71) Applicant: CERNER INNOVATION, INC., KANSAS CITY, KS (US)

(72) Inventors: Huma Rahman, Bengaluru (IN); Pagi Prashant, Bengaluru (IN); Medha Shukla, Agra (IN)

(21) Appl. No.: 17/366,647

(22) Filed: Jul. 2, 2021

(51) Int. Cl. G16H 40/20; G16H 10/60; G16H 50/70; G16H 50/30; G07C 9/37; G07C 9/38; G16H 40/67; G16H 50/20; G06F 16/35; G06F 40/205; G06K 9/00; A61B 5/0205; A61B 5/021; A61B 5/08; A61B 5/145; A61B 5/00; A61B 5/024

(52) U.S. Cl. CPC G16H 40/20; G16H 10/60; G16H 50/70; G16H 50/30; G07C 9/37; G07C 9/38; G16H 40/67; G16H 50/20; G06F 16/35; G06F 40/205; G06K 9/00288; A61B 5/02055; A61B 5/021; A61B 5/0816; A61B 5/14542; A61B 5/4824; A61B 5/024

(57) ABSTRACT

Systems and methods for intelligently and accurately triaging a patient are provided. The systems and methods access an electronic health record of the patient based on a detection of an event, such as entering an emergency department. Additionally, the systems and methods collect triage vitals of the patient. Further, free-text within the electronic health record is mapped and binned. In response to the mapping and the binning, the systems and methods input features into an acuity level predictor. Based on inputting the features into the acuity level predictor, the systems and methods output an acuity level of the patient for triaging the patient.

### BACKGROUND

Triage is the process of determining a priority of a patient to be treated based on a condition of the patient and a likelihood of recovery with and without treatment. Triage committees and ad hoc consultative bodies make decisions on how to triage incoming patients. A goal of triaging is to save as many lives as possible, while factoring in resources and personnel. For example, clinicians may determine that resources required for a patient with a low likelihood of survival be diverted to others with higher likelihoods of survival to increase their chances of surviving.

Triage is the first clinical process performed on the patient upon arrival. Typically, patients are first categorized by acuity to prioritize individuals who require urgent medical care. Subsequently, a clinician will attend to the patient. The clinician will create an initial care plan and recommend an approach. Quickly and effectively triaging patients allows for prompt clinical intervention.

### SUMMARY

At a high level, aspects described herein relate to intelligently and accurately triaging a patient. In aspects, based on the detection of an event, a computerized method accesses an electronic health record of the patient and collects triage vitals of the patient. The computerized method maps and bins free-text from the electronic health record and the triage vitals into categorical features using natural language processing. Further, the computerized method transforms numerical features from the electronic health record and the triage vitals using a statistical imputation. Additionally, the computerized method inputs the categorical features that were binned and the numerical features that were transformed into an acuity level predictor. Based on inputting the features into the acuity level predictor, the computerized method outputs an acuity level of the patient for triaging the patient.

In another aspect, non-transitory computer storage media cause a processor to perform operations for intelligently and accurately triaging a patient. The operations comprise accessing an electronic health record of the patient and collecting triage vitals of the patient based on a detection of an event. The operations also comprise mapping and binning free-text from the electronic health record and the triage vitals into categorical features using natural language processing for input into an acuity level predictor. Additionally, the operations comprise inputting the categorical features into the acuity level predictor that uses a machine learning algorithm for recalculating thresholds at each level of a decision tree. Each level of the decision tree corresponds to one of the categorical features. Based on inputting the categorical features into the acuity level predictor, the operations additionally comprise outputting an acuity level of the patient for triaging the patient.

In yet another aspect, an intelligent triage system triages a patient. The intelligent triage system comprises a processor and one or more computer storage media storing computer-executable instructions embodied thereon that when executed by the on processor, cause the processor to perform operations. For example, the intelligent triage system retrieves an electronic health record of the patient and collects triage vitals based on a detection of an event. Additionally, the intelligent triage system maps and bins free-text from the electronic health record and the triage vitals into categorical features using natural language processing for input into an acuity level predictor. Further, the intelligent triage system inputs the categorical features into the acuity level predictor that recalculates a threshold at each level of a decision tree. Each level of the decision tree corresponds to one of the categorical features. Based on inputting the categorical features into the acuity level predictor, the intelligent triage system outputs an acuity level of the patient for triaging the patient.

This summary is intended to introduce a selection of concepts in a simplified form that is further described in the Detailed Description section of this disclosure. The Summary is not intended to identify key or essential features of the claimed subject matter, nor is it intended to aid in determining the scope of the claimed subject matter. Additional objects, advantages, and novel features of the technology are included in the Detailed Description, and in part, will become apparent to those skilled in the art upon examination of the disclosure or learned through practice of the technology.

### BRIEF DESCRIPTION OF THE DRAWINGS

The present technology is described in detail below with reference to the attached drawing figures, wherein:

FIG. 1 is an example flowchart for determining an acuity level, in accordance with an aspect described herein;

FIGS. 2A-2B provide example tables comprising data for preparation to input features from the data into an acuity level predictor, in accordance with an aspect described herein;

FIG. 3 illustrates an example computational decision tree for determining an acuity level of a patient being triaged, in accordance with an aspect described herein;

FIG. 4 illustrates an example flowchart for determining treatment based on an acuity level, in accordance with an aspect described herein;

FIG. 5 illustrates an electronic triage powerform chart, in accordance with an aspect described herein;

FIG. 6 illustrates an example emergency room launchpoint, in accordance with an aspect described herein;

FIG. 7 illustrates an example emergency department tracking board, in accordance with an aspect described herein;

FIG. 8 illustrates a schematic diagram for automated triaging, in accordance with an aspect described herein;

FIG. 9 includes a flowchart for intelligently and accurately triaging a patient via an automated process, in accordance with an aspect described herein; and

FIG. 10 is an example computing environment suitable for implementing aspects of the disclosed technology, in accordance with an aspect described herein.

### DETAILED DESCRIPTION

The subject matter of the present technology is described with specificity herein to meet statutory requirements. However, the description itself is not intended to limit the scope of this disclosure. Rather, the inventors have contemplated that the claimed or disclosed subject matter might also be embodied in other ways, to include different steps or combinations of steps similar to the ones described in this document, in conjunction with other present or future technologies. Moreover, although the terms “step” or “block” might be used herein to connote different elements of methods employed, the terms should not be interpreted as implying any particular order among or between various steps herein disclosed unless and except when the order of individual steps is explicitly stated.

For purposes of this disclosure, the word “including” has the same broad meaning as the word “comprising,” and the word “accessing” comprises “receiving,” “referencing,” or “retrieving.” Further the word “communicating” has the same broad meaning as the word “receiving,” or “transmitting” facilitated by software or hardware-based buses, receivers, or transmitters” using communication media described herein. Also, the word “initiating” has the same broad meaning as the word “executing or “instructing” where the corresponding action can be performed to completion or interrupted based on an occurrence of another action.

In addition, words such as “a” and “an,” unless otherwise indicated to the contrary, include the plural as well as the singular. Thus, for example, the constraint of “a feature” is satisfied where one or more features are present. Also, the term “or” includes the conjunctive, the disjunctive, and both (a or b thus includes either a or b, as well as a and b).

For purposes of a detailed discussion above, embodiments of the present technology described with reference to a distributed computing environment; however, the distributed computing environment depicted herein is merely an example. Components can be configured for performing novel aspects of embodiments, where the term “configured for” or “configured to” can refer to “programmed to” perform particular tasks or implement particular abstract data types using code.

As will be described, one or more methods, computer storage media, and systems for intelligently triaging a patient are provided. In the clinical setting, it is difficult to quickly and accurately triage a patient. For example, conventional systems and methods inaccurately triage patients due to an under-evaluation of a high acuity and/or an overestimation of urgency without any severe illness. The overestimation and under-evaluation can result from unaccounted for information, such as particular patient vitals taken during triage and previous clinical data pertaining to the triaged patient stored in an electronic health record. High volumes of patients entering an emergency department for triage may also contribute to the overestimation and under-evaluation of urgency.

Additionally, the conventional systems and methods are slow and have latency issues due to the high volumes and due to reliance on clinicians making decisions for acuity determinations. The acuity determinations made by the conventional methods and systems fail to account for hidden patterns within large volumes of historical clinical data stored, which would enhance accuracy. Further, the acuity determinations of the conventional methods and systems do not analyze and transform input variables, which would enhance the speed and inhibit the latency issues. The conventional systems and methods fail to utilize the decisions for acuity determinations for forecasting patient dispositions and estimating resource utilization for critical care, radiology and laboratory services, intensive care unit admission, and the like.

To solve these problems with the conventional systems and methods, the present aspects described herein provide for improved patient triaging. In aspects, based on the detection of an event, a computerized method accesses an electronic health record of the patient and collects triage vitals of the patient. The computerized method maps and bins free-text from the electronic health record and the triage vitals into categorical features using natural language processing. Further, the computerized method transforms numerical features from the electronic health record and the triage vitals using a statistical imputation. Additionally, the computerized method inputs the categorical features that were binned and the numerical features that were transformed into an acuity level predictor. Based on inputting the features into the acuity level predictor, the computerized method outputs an acuity level of the patient for triaging the patient.

In an embodiment, non-transitory computer storage media cause a processor to perform operations for intelligently and accurately triaging a patient. The operations comprise accessing an electronic health record of the patient and collecting triage vitals of the patient based on a detection of an event. The operations also comprise mapping and binning free-text from the electronic health record and the triage vitals into categorical features using natural language processing for input into an acuity level predictor. Additionally, the operations comprise inputting the categorical features into the acuity level predictor that uses a machine learning algorithm for recalculating thresholds at each level of a decision tree. Each level of the decision tree corresponds to one of the categorical features. Based on inputting the categorical features into the acuity level predictor, the operations additionally comprise outputting an acuity level of the patient for triaging the patient.

In yet another embodiment, an intelligent triage system triages a patient. The intelligent triage system comprises a processor and one or more computer storage media storing computer-executable instructions embodied thereon that when executed by the on processor, cause the processor to perform operations. For example, the intelligent triage system retrieves an electronic health record of the patient and collects triage vitals based on a detection of an event. Additionally, the intelligent triage system maps and bins free-text from the electronic health record and the triage vitals into categorical features using natural language processing for input into an acuity level predictor. Further, the intelligent triage system inputs the categorical features into the acuity level predictor that recalculates a threshold at each level of a decision tree. Each level of the decision tree corresponds to one of the categorical features. Based on inputting the categorical features into the acuity level predictor, the intelligent triage system outputs an acuity level of the patient for triaging the patient.

Implementation of the embodiments above provides for faster and more accurate patient triaging without having to rely upon human subjective determinations. Additionally, implementation of the embodiments result in more accurate determinations of acuity with less under-evaluations and overestimations of urgency without any severe illness. Further, these implementations result in reduced wait time for patient treatment, improved queuing of patients, improved qualities of emergency department services, decreased morbidity, improved emergency department throughput, improved boarding, decreased errors in patient treatment, and overall improved qualities of care.

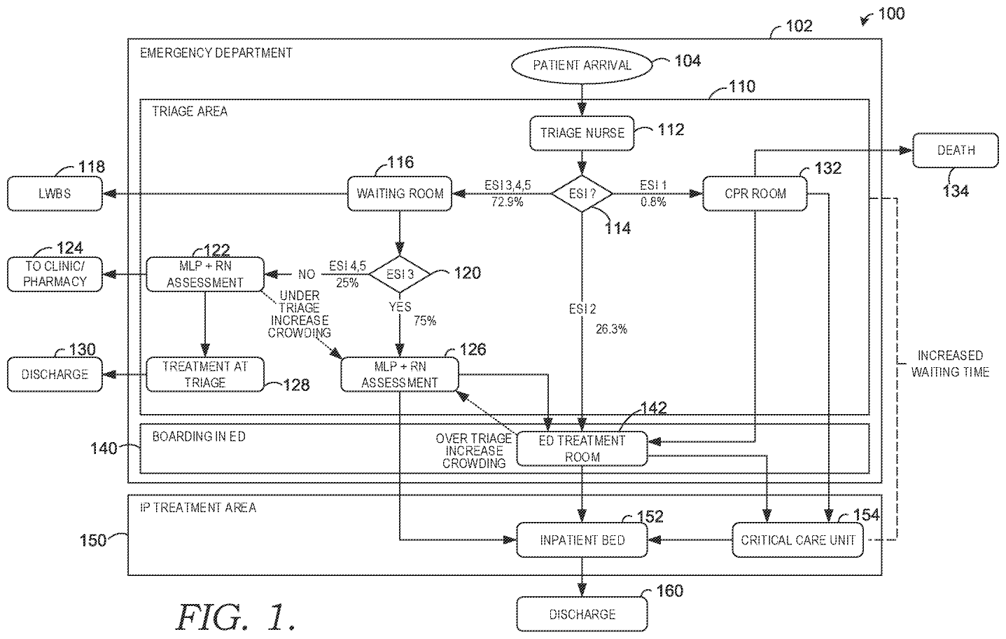

Referring now to the drawings, FIG. 1 illustrates an example emergency department workflow 100 based on acuity level. An acuity level predictor 114 determines an acuity level by using a machine learning algorithm and patient data comprising historical clinical data stored in an electronic health record and vitals that are collected and measured during triage of the patient. The acuity level predictor 114 provides decision support by predicting acuity and the likelihood of radiology services, intensive care unit admissions, and inpatient hospitalizations. The electronic health record may include data from various electronic health record systems that are communicatively coupled to a network. For example, the electronic health record systems may comprise electronic health records from a hospital, a health information exchange, and an ambulatory clinic. Additionally, other electronic health records may include records from a mental health facility and a behavioral health facility.

Referring back to the acuity level, a five-level triage algorithm may include the following example acuity levels:

TABLE 1
Acuity Levels

| Acuity Level | Health Deterioration |
|---|---|
| Level 1 | Resuscitation |
| Level 2 | Emergent |
| Level 3 | Urgent |
| Level 4 | Less Urgent |
| Level 5 | Non-Urgent |

Referring to Table 1, a patient who has an acuity level 1 would have a higher need for particular resources than a patient who has an acuity level of 3-5. For example, a patient having acuity level 1 may require immediate intervention to save her life or to save her limb. As another example, a patient having acuity level 2 may have a high risk for requiring a treatment sensitive to time. Further, patients who are not assigned to the first two levels are assigned to levels 3-5. Resource availability has an impact on which level a patient is assigned. In some embodiments, a patient at level 3 requires two or more resources, at level 4 requires one resource, and at level 5 requires no resources.

Conventional systems have nurses or other humans determining acuity levels for patients. For example, nurses would determine which facilities are required for the patient and determine an acuity level based on the facilities required. By contrast, the acuity level predictor 114 provides for more accurate acuity levels based on hidden patterns in large volumes of historical clinical data that conventional systems failed to detect and utilize. As a result, the acuity level predictor 114 improves resource distribution (e.g. in the emergency department), more effectively ques patients, and decreases morbidity by providing rapid and informed decisions that enhance resource planning.

As such, example emergency department workflow 100 provides for an example triaging scheme based on acuity level. Example emergency department workflow 100 comprises an emergency department 102, triage area 110, boarding in the emergency department 140, and inpatient (IP) treatment area 150. Upon patient arrival 104 at the emergency department 102, a triage nurse 112 attends to the patient within the triage area 110. The triage nurse 112 may enter the name of the patient and/or other identification information into a computing device that is in communication with one or more electronic health record systems comprising one or more electronic health records.

Additionally, the triage nurse 112 may take the patient's vitals. For example, vitals may include temperature, oxygen saturation, blood pressure, respiratory rate, heart rate, and so forth. In some embodiments, the nurse acquires the vitals via one or more devices. In some embodiments, vitals are captured via a video device, an audio device, heat sensors, facial imaging devices, and/or other detection devices. In some embodiments, vitals may be captured via an x-ray radiology device that collects information audibly, visually, and/or electronically available within a range of the x-ray radiology device. Continuing the example, a communication interface (e.g., Wi-Fi, near field communication, universal serial bus, Ethernet, open natural interaction, natural user interface, etc.) is included in or with the x-ray radiology device for capturing environmental data and other patient information in addition to image data captured via the x-ray radiology device.

Using the electronic health records corresponding to the patient and the vitals, the acuity level predictor 114 determines an acuity level. In some embodiments, the acuity level scale ranges from 1-5, such as the Emergency Severity Index (ESI). For acuity levels 3-5, those patients enter the waiting room 116. In some embodiments, 72.9% of all patients who enter the emergency department 102 receive an acuity level of 3, 4, or 5. For the patients with an acuity level of 3, 4, or 5, those patients have a lower risk of health deterioration while waiting for emergency department services than patients with an acuity level of 1 or 2. By improving inaccurate triaging based on human error that leads to adverse events, acuity level predictor 114 improves patient safety and care quality at least in part by reducing this error.

Further, conventional systems that introduce more error result in patients leaving without being seen (LWBS) 118. For example, studies suggest that patients that nurses incorrectly triaged as an ESI level 3, but should have been triaged as an ESI level 2, will LWBS 118. Continuing the example, some studies suggest that up to 80% of patients who enter an emergency department and sit in the waiting room will LWBS 118. As such, acuity level predictor 114 provides for improved allocation of services by more accurately triaging patients.

In some embodiments within the triage area 110, patients assigned an acuity level of 4 or 5 via the acuity level predictor at step 120 are further assessed at 122 by registered nurses (RNs) and/or mid-level practitioners (MLPs) who have a defined scope of practice. Continuing the example, in some embodiments, 25% of the 72.9% of patients (having an acuity level 3-5) have an acuity level of 4 and 5. Based on the assessment at 122 by the MLP and/or the RN, the patients having the acuity level of 4 and 5 are either sent to a clinic or a pharmacy at 124 or receive treatment at 128. Those who receive treatment at 128 are subsequently discharged at 130. Patients who were under-triaged as 4 and 5, but should have been triaged as a lower acuity level, increase the crowding in the waiting room 116 and increase the wait time for services. For example, patients who were under-triaged by a conventional system would have needed treatment from the emergency department treatment room 142 or would have needed an inpatient bed 152, but instead were sitting in the waiting room 142.

In some embodiments within the triage area 110, patients assigned an acuity level of 3 via the acuity level predictor at step 120 are further assessed at 126 by an MLP and/or an RN. Continuing the example, in some embodiments, 75% of the 72.9% of patients (having an acuity level 3-5) have an acuity level of 3. Upon assessment at 126, the patients having the acuity level of 3 are either sent to the emergency department treatment room at 142 within boarding in the emergency department 140, or are sent to the inpatient bed at 152 within the IP treatment area 150. In some embodiments, the patient moves from the emergency department treatment room at 142 to the critical care unit 154. Continuing the example, after the critical care unit 154, the patient may move to the inpatient bed at 152 and thereafter be discharged 160.

For a conventional system that over-triages patients, the result of this over-triage includes increased crowding at the emergency department room 142. For example, patients who were over-triaged as 3, but should have been triaged as a 4 or a 5, increase crowding during boarding in the emergency department 140 and increase the wait time for emergency department services for patients actually having a greater need for those services. As such, acuity level predictor 114 provides for improved allocation of services by improving boarding in the emergency department 140 and by making boarding in the emergency department 140 more efficient due to more accurately triaging patients.

Returning to acuity level predictor 114, in some embodiments, the acuity level predictor 114 assigns 26.3% of the patients who enter the emergency department 102 an acuity level 2. As illustrated in workflow 100, the patients who are assigned an acuity level 2 receive boarding in the emergency department 140 for the emergency department treatment room 142. Thereafter, the patient is processed in the IP treatment area 150 for an inpatient bed 152 or a critical care unit 154. If assigned to the critical care unit 154, the patient may then be assigned to the inpatient bed 152. After the inpatient bed 152, the patient may be discharged 160.

Further, in some embodiments, the acuity level predictor 114 assigns 0.8% of the patients who enter the emergency department 102 an acuity level 1. These patients assigned acuity level 1 are sent to a cardiopulmonary resuscitation (CPR) room 132. From there, the patients either die 134 or are then sent to the emergency department treatment room 142. Thereafter, the patient is processed in the IP treatment area 150 for an inpatient bed 152 or a critical care unit 154. If assigned to the critical care unit 154, the patient may then be assigned to the inpatient bed 152. After the inpatient bed 152, the patient may be discharged 160.

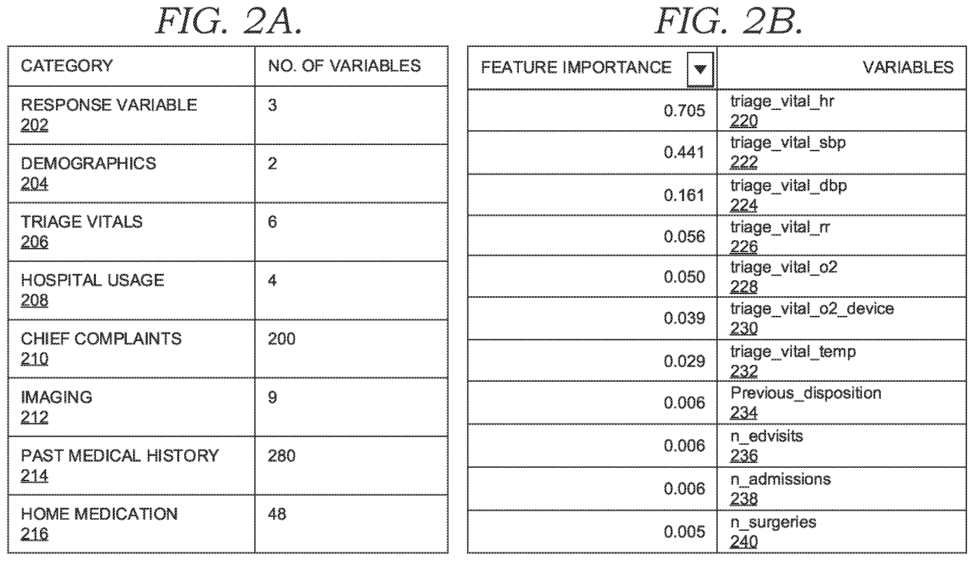

Turning now to FIGS. 2A-2B, FIGS. 2A-2B provide example tables comprising data prepared as input features into an acuity level predictor. As illustrated in FIG. 2A, the data for preparation may comprise a response variable 202, demographics 204, triage vitals 206, hospital usage 208, chief complaints 200, imaging 212, past medical history 214, and home medication 216. The data for preparation may be stored in the electronic health record. Beginning with response variable 202, include predicted acuity levels by the acuity level predictor for a particular patient or for a sample population of patients.

Turning now to demographics 204, demographics 204 may include an insurance provider, an insurance card grouping number, provider information for specialists, primary care provider information, date of birth, age, gender, ethnicity, country of origin, first name, family name, middle name(s), home address, work address, etc. Additionally, triage vitals 206 may include body temperature, blood pressure, heart rate, respiratory rate, blood oxygen level, a pain score (e.g., a numerical rating scale, a visual analog scale, a categorical scale), etc. In addition, hospital usage 208 may include a number of emergency department visits within a period of time (e.g., a year or a lifetime), a number of admissions into a particular hospital, a number and types of surgeries performed on the patient within a period of time (e.g., a year or a decade), and a number and types of procedures performed on the patient within a period of time.

Turning to chief complaints 210, chief complaints 210 may include a problem on admission, an initial comment to a clinician, an observed or detected degree of severity, and observed or detected symptoms. Further, examples of imaging 212 include a number of chest x-rays, an echocardiogram, a computerized tomography scan, magnetic resonance imaging, and medical ultrasound. In some embodiments, the imaging 212 may only include imaging 212 within the past year. Additionally, examples of past medical history 214 include information related to diet and exercise, allergy information, immunization history, family medical history, social history, relevant chronic illnesses, prior diseases, etc. Turning to home medications 216, home medications 216 may include active home medications, an amount of a strength of the medication taken within a period of time, a method of administration of the medication, etc.

Preparation of the data (e.g., the response variable 202, the demographics 204, the triage vitals 206, the hospital usage 208, the chief complaints 200, the imaging 212, the past medical history 214, and the home medication 216) may include mapping and binning. For example, data from the chief complaints 210 stored in the electronic health record are extracted and binned into the “chief complaints” 210 category. Additionally, data within the chief complaints 210 category are further mapped and binned into a plurality of categorical features. For example, the chief complaints having an observed or detected degree of severity of 8-10 are mapped and binned into one category feature, and the chief complaints having the observed or detected degree of severity of 4-6 are mapped and binned into a second category feature.

Further, the data (e.g., the free-text) from the chief complaints 210 category may be reduced to binary variables (e.g., a high detected degree of severity 1, a low detected degree of severity 0; admitted patient was unconscious 1 (yes), admitted patient was not unconscious (0)) by binning frequent data in a plurality of categories. As another example, ICD-9 codes corresponding to the past medical histories 214 may be mapped to 281 categories by binning to a binary variable. In yet another example, features of the active home medications 216 may be binned into 48 therapeutic subgroups (e.g., analgesics) based on values that are most frequent. Each category may comprise a number of variables.

In some embodiments, a pattern classifier may determine one or more patterns of the mapped and binned information. Further, the classifier may also determine one or more patterns corresponding to the binary variables. The classifier may include a plurality of classifiers. Further, a single software module may implement the classifier or the plurality of classifiers. Patterns in both numerical values and free-text may be determined. In some embodiments, a change to a knowledge representation caused by training may only require duplication of one model. Pattern(s) determined may be used for further determinations of an feature importance corresponding to a respective category.

In some embodiments, for example, numerical features (e.g., temperature, blood pressure, respiratory rate, pain score, heart rate, age, number of hospital visits in the past 1 year, number of surgeries/procedures in past 1 year, etc.) are transformed by null handling using median and mean imputation. For example, median and mean imputation may be determined from electronic health record data corresponding to a plurality of patients having predetermined similarities (e.g., data from patients within a particular age range having a similar number of the same procedure, a similar body weight, a similar height, and a similar city of origin may be used for missing value imputations). In some embodiments, the median and mean imputation may be determined from historical electronic health record data corresponding to the patient being triaged. The median and mean imputation(s) may be used for further determinations of the feature importance corresponding to the respective category.

In some embodiments, natural language processing (NLP) software maps and bins free-text from the electronic health record and the triage vitals into categories and categorical features. Machine learning algorithms may include supervised learning based on mapping variables, from information for a plurality of patients having a known ESI, to a fixed set of labels based on the known ESI. For example, the NLP software extracts the information for the plurality of patients having the known ESI from an open-source website having a plurality of emergency department electronic health records. In another embodiment, the NLP software extracts the information for the plurality of patients having the known ESI from a plurality of hospital electronic health records. In some embodiments, the information is extracted from one system corresponding to an emergency department.

Turning to FIG. 2B, the left column includes a feature importance and the right column corresponds to the variable. For example, the triage vital heart rate 220 has a feature importance of 0.705, the triage vital systolic blood pressure 222 has a feature importance of 0.441, the triage vital diastolic blood pressure 224 has a feature importance of 0.161, the triage vital respiratory rate 226 has a feature importance of 0.056, the triage vital oxygen saturation 228 has a feature importance of 0.050, the triage vital oxygen saturation via pulse oximetry 230 has a feature importance of 0.039, the triage vital temperature 232 has a feature importance of 0.029, the triage vital corresponding to a previous disposition of the patient 234 has a feature importance of 0.006, the triage vital number of emergency department visits 236 has a feature importance of 0.006, the triage vital number of admissions 238 has a feature importance of 0.006, and the triage vital number of surgeries 240 has a feature importance of 0.005.

One or more processors may determine the feature importance using a decision tree. For example, the feature importance may be determined using a Gini index (an impurity measure for determining the split at an internal node of a decision tree, such as a random forest) and an entropy gain. For example, higher feature importance scores indicate that the corresponding feature is more predictive than the lower feature importance scores. The feature importance of each feature may be determined based on a total decrease in node purity averaged over each tree in the forest. By using the categories from the mapping and the binning for the decision tree, the decision tree is better able to perform classification or regression tasks without undue increase in computational load.

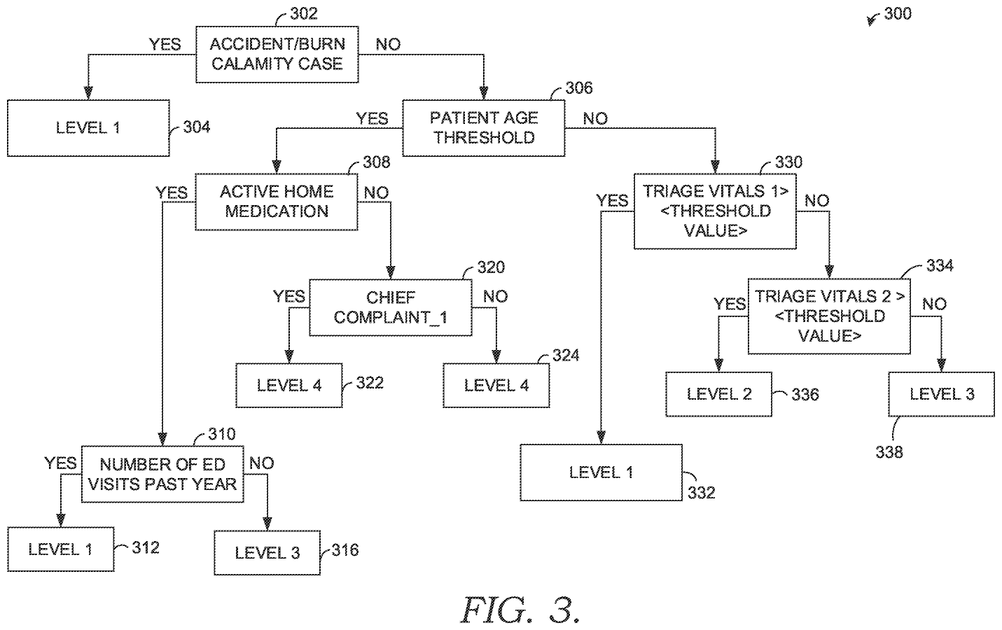

Turning to FIG. 3, FIG. 3 illustrates an example decision tree 300 for a computing system that determines an acuity level of a patient being automatically and intelligently triaged. The size and shape of the decision tree depends on various factors, such as the number of independent variables that are found to be significant for predictions based on the feature importance scores. Further, mapping and binning of categories and determinations of the feature importance scores dictate the order in which a tree-generation algorithm analyzes the category features, numerical features, and so on. In some embodiments, the decision tree 300 may require or rely on ensembles or collections of many different trees (e.g., respective trees obtained using respective subsets of training data sets).

At decision node 302, if the patient being triaged includes an accident/burn calamity case, then the acuity level predictor gives the patient an acuity level of 1 at 304. If the patient is not an accident/burn calamity case, then decision node 306 determines whether the patient has an age that is above a threshold (e.g., a patient who is 80 or older). If the patient is at an age over the threshold, then decision node 308 determines whether the patient has a particular active home medication (e.g., a medication with a particular active ingredient). If the patient has the particular active home medication, then decision node 310 determines whether the patient has had a particular number of emergency department visits in the past year. If the number of emergency department visits exceeds a threshold, then the acuity level predictor gives the patient an acuity level of 1 at 312. If the number of emergency department visits does not exceed the threshold, then the acuity level predictor gives the patient an acuity level of 3 at 316.

Turning back to 308, if the decision node determines that the patient does not have the particular active home medication, then decision node 320 determines whether the patient has a particular chief complaint or a particular feature of the particular chief complaint. If the patient has the particular chief complaint or the particular feature, then the acuity level predictor gives the patient an acuity level of 4 at 322. If the patient does not have the particular chief complaint or the particular feature, then the acuity level predictor gives the patient an acuity level of 4 at 322. Continuing the example, the particular chief complaint or the particular feature of the particular chief complaint may have a feature importance score of 0.005, which is lower than the other feature importance scores.

Turning back to 306, if the patient is at an age that is not over the threshold, then if the patient has other triage vitals that the acuity level predictor determines are at level 1, then the patient receives an acuity level of 1 at 332. If the patient does not have the other triage vitals, then the decision node 334 determines whether the patient has a triage vital that the acuity level predictor determines is level 2. If the patient has these level 2 triage vitals, then the patient receives an acuity level of 2 at 336. If the patient does not have these level 2 triage vitals, then the patient receives an acuity level of 3 at 338.

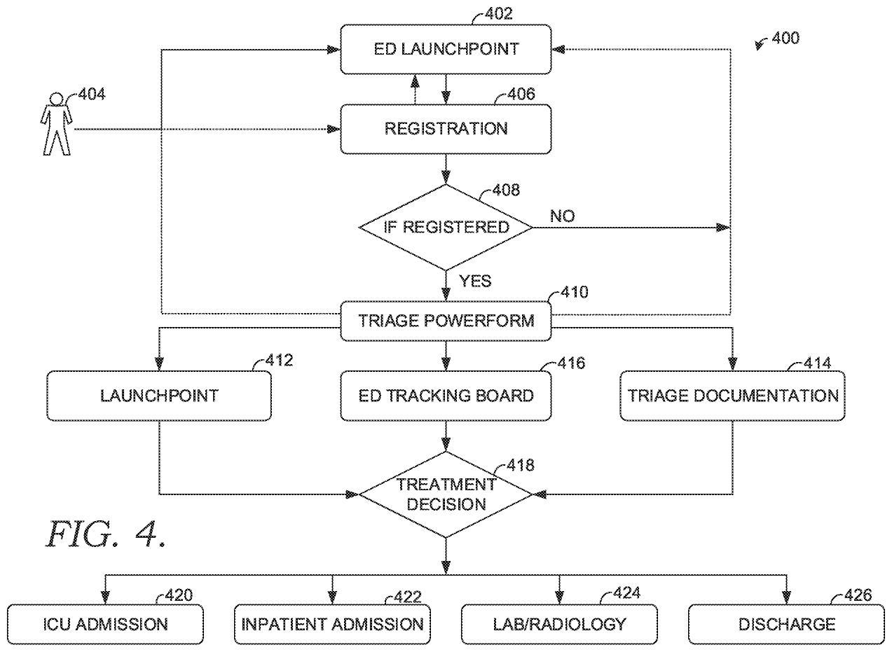

Turning to FIG. 4, FIG. 4 illustrates an example flowchart 400 for patient interaction with a predetermined solution within the emergency department. At the emergency department, a patient 404 enters the emergency room and data is immediately reflected within a launchpoint dashboard 402. At 406, the patient 404 is automatically or manually registered with a computing system in communication with at least one electronic medical record of the patient 404. If the patient 404 is not automatically registered at 408, the system may require manual entry of the patient 404. launch point 402 displays the data captured during the registration. Upon registration, triage powerform 410 is charted by a triage nurse. After the triage powerform 410 is signed and submitted, the system initiates a launchpoint 412. The data is then reflected on launchpoint 412, triage documentation 414, and an emergency department tracking board 416. Launchpoint 412 initiates the emergency department workflow from triage through discharge. The triage documentation 414 automatically provides templates for documentation based on symptoms upon entry and/or age and gender. The documentation may be stored in a database associated with the electronic health record system. The emergency department tracking board 416 provides for tracking a status of the patient and monitoring resource availability.

The triage powerform 410 may receive manual information including an oral temperature, heart rate, respiratory rate, and blood pressure. In some embodiments, triage powerform 410 may automatically populate an onset date and time, a mode of arrival, and a reason for visit. In some embodiments, triage powerform 410 automatically populates vitals using sensors (e.g., a thermal imaging camera or infrared pyrometer). In some embodiments, triage powerform 410 automatically populates a weight and height of the patient by extracting the information from an electronic medical record of the patient.

As an example, a clinician may enter into a chief complaint or mechanism of injury entry area that that patient has severe abdominal pain, is vomiting, and has nausea. Continuing the example, another entry area may include a reason for not obtaining current visit information, which may include a list for selection of the following: none, clinical condition, cognitive impairment, intubated, language barrier, physical impairment, sedated, and other. Additionally, another entry area may include demonstrating signs and symptoms of the following condition, which may include a list for selection of the following: acute coronary syndrome, asthma, heart failure, pneumonia, stroke, and venous thromboembolism. In some embodiments, triage powerform 410 automatically populates allergy information of the patient by extracting the information from the electronic medical record of the patient.

Based on categorical features and numerical features corresponding to data received by the triage powerform 410 and data extracted by the triage powerform 410 from the electronic health record, an acuity level of the patient for triaging is determined. Based on the acuity level, a treatment decision 418 is provided. For example, treatment decision 418 may present for display a tracking group that the patient was grouped into (e.g., grouping based on a level of cognition when entering an ambulatory vehicle or the emergency department). As another example, the treatment decision 418 may present for display the acuity level (e.g., emergent).

Examples of an acuity level 1 may include immediate life-saving intervention needed, including airway medications, other hemodynamic interventions, and any of the following clinical conditions: intubated, apneic, pulseless, severe respiratory distress, SpO₂ less than 90%, acute mental status changes, unresponsiveness (e.g., nonverbal or requires noxious stimulus). An example of an acuity level 2 may include high risk situations wherein a patient is in severe pain or distress and has a pain score of 7 or higher on a pain scale of 0-10. Further, an example of an acuity level 3 may include a need for electrolytes/coagulations and a chest x-ray. An example of an acuity level 4 may include a need for electrolytes/coagulations (i.e., one resource). Additionally, an example of an acuity level 5 may include a patient who does not need any resources.

The treatment decision 418 may provide an acuity level and a recommended treatment procedure based on whether the patient needs an immediate life-saving intervention; whether the patient is confused, lethargic, disoriented, in severe pain, and/or in severe distress; the number of resources the patient needs; and/or vital signs that the patient has or is exhibiting. The recommended treatment procedure may include ICU admission 420, inpatient admission 422, lab/radiology testing 424, and discharge 426. For example, the launchpoint 412 may display an outcome predictor that provides a predicted probability that the patient will need the ICU admission 420, the inpatient admission 422, and/or the lab/radiology testing 424. In some embodiments, in response to providing the acuity level and/or the recommended treatment(s), the system provides a selection to accept the acuity level predicted. Upon providing the selection, a user may select to accept or override (e.g., upon the entry of improper data) the acuity level predicted.

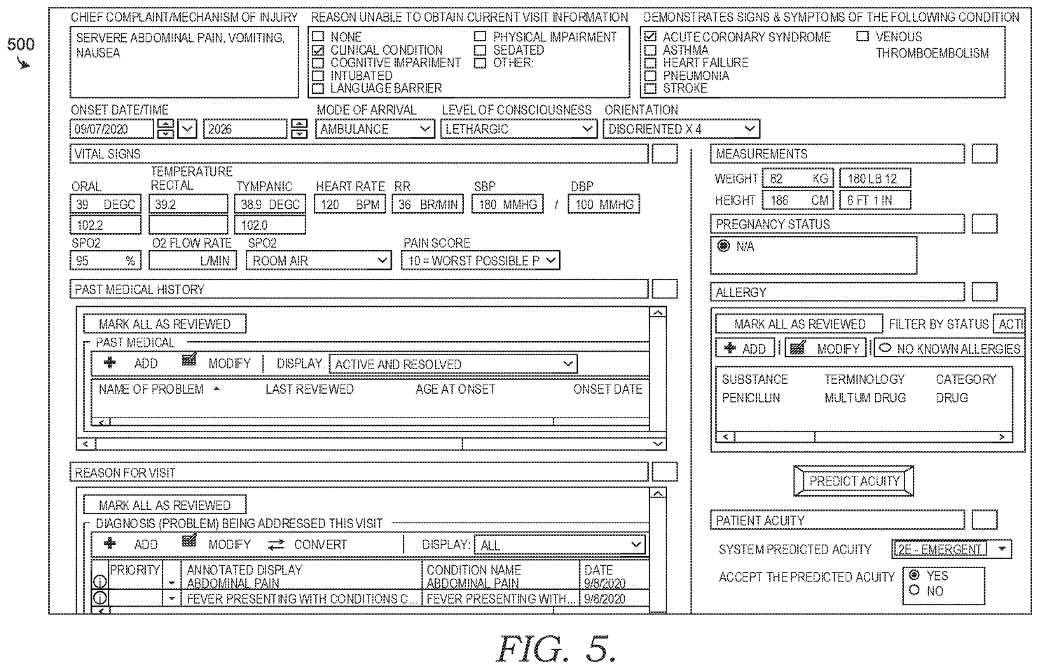

Turning FIG. 5, an example selectable, editable electronic triage powerform chart 500 is illustrated. Powerform chart 500 comprises editable sections including a chief complaint or mechanism of injury, a reason that the system was unable to obtain current visit information, signs and symptoms of various conditions, an onset date and time, a mode of arrival, a level of consciousness, and an orientation. The editable selections may be automatically populated by the electronic system in some embodiments. For example, a device comprising video and audio sensors may detect and determine that the patient is vomiting in the ambulance or the emergency room. Further, other editable selections may include vital signs, weight and height, a pregnancy status, allergy information, past medical history, a reason for visit, and problems being addressed during the visit. An acuity level may be predicted upon selection of an acuity button. Further, a selection to accept or not to accept the acuity prediction may be received.

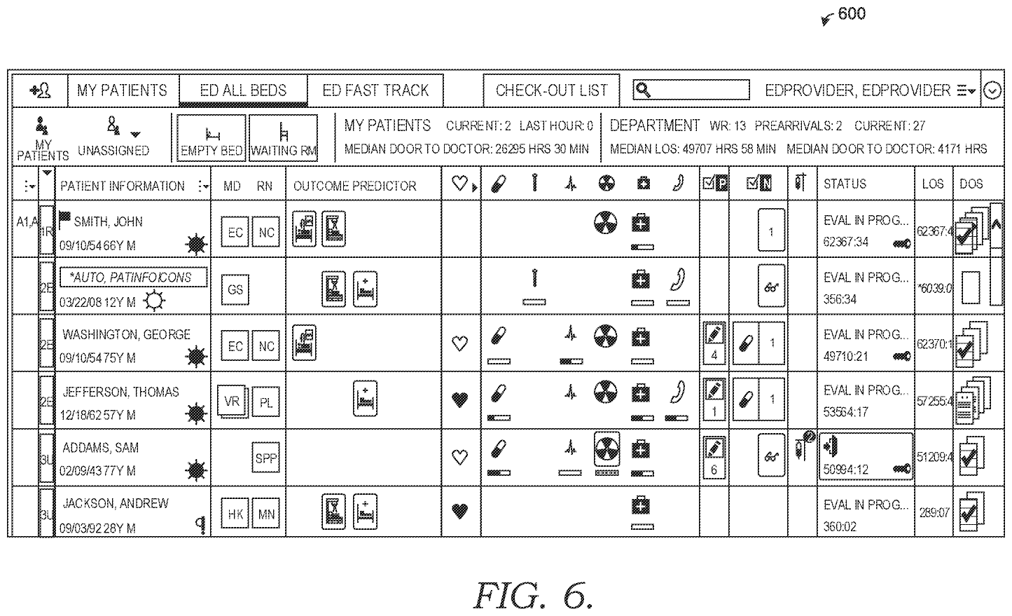

Turning to FIG. 6, example emergency room launchpoint 600 comprises tabs for patients corresponding to a particular provider of care in the emergency department, bed availability, patients on a fast track to discharge, and a check-out list. Each tab may provide a list of “my patients,” “unassigned patients,” “empty beds,” “patients in the waiting room,” and “department information.” Further, the emergency room launchpoint 600 also includes patient information, information about the nurses and doctors attending to the patient, and outcome predictors. Outcome predictors use the proposed acuity level from the acuity level predictor and to estimate, for example, whether the patient will need imaging, advanced diagnostic imaging, an ICU bed, inpatient admission, other critical care resources, and so forth.

In embodiments, a learning classification/regression model capable of improving accuracy of the classifications via labeling categorical features without increasing computational overhead for learning and classifying. For example, the classification regression model may be trained using training data and labeling pairs for predicting an outcome. Labeling may correspond to categories (e.g., chief complaints and active home medications), subcategories (e.g., 48 therapeutic subgroups of the active home medications), and one or more corresponding acuity levels. Training data may correspond to acuity levels correctly determined for a plurality of patients from one or more electronic health records. In embodiments, during the learning process, a computing system lists combinations of labels appearing together in one or more training data to obtain a combination of labels expected to appear together for an input.

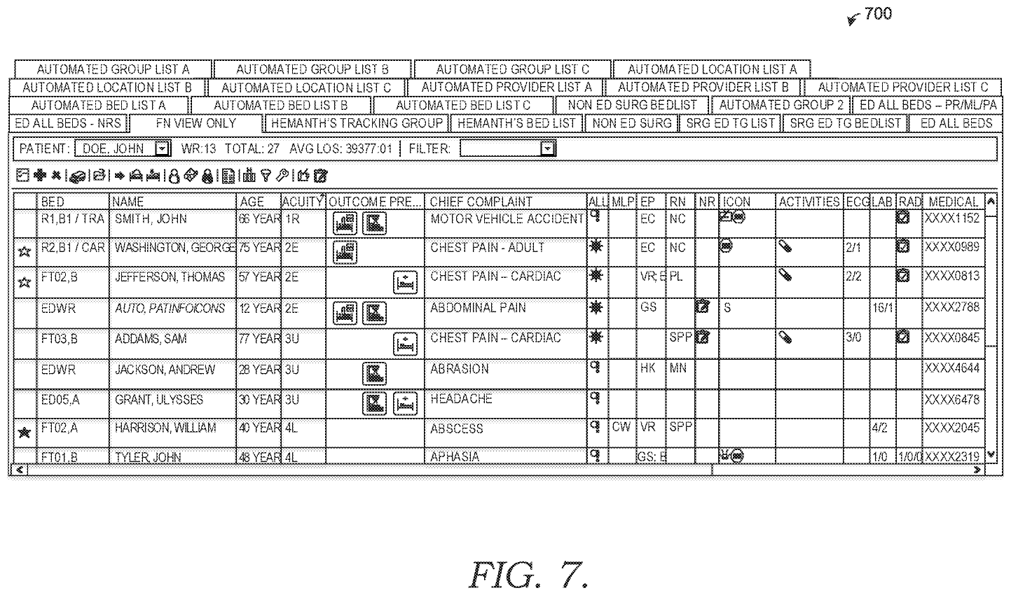

Turning now to FIG. 7, example emergency department tracking board 700 comprises patient information including name, bed location, age, a predicted acuity level, outcome predictions, a chief complaint, attending clinician information, and a medical identification. Further, example emergency department tracking board 700 provides warning icons for patients who need immediate assistance. Additionally, a number of patients in the waiting room is provided. Further, other icons for radiology and medications are illustrated on the emergency department tracking board 700. The radiology predictor icon provides for rapid identification of patients who require radiology imaging during their emergency department visit. The predictor icons assist clinicians in identifying those with a higher risk of ICU admission. Further, the predictor icons help reduce emergency department ICU wait time. Furthermore, the emergency department tracking board 700 helps clinicians to manage resources more efficiently to reduce boarding errors and delays.

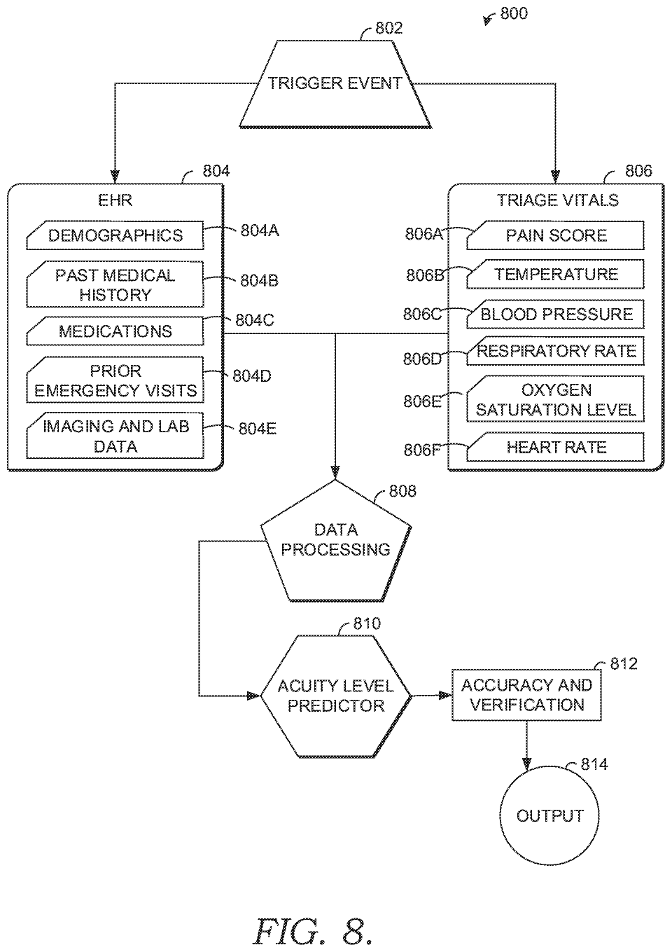

Turning to FIG. 8, FIG. 8 is a schematic diagram 800 for automated triaging. Triaging begins upon the occurrence of a trigger event 802, such as a patient entering an emergency department or an ambulatory vehicle. In some embodiments, the triggering event is a video call from a patient to an emergency department. In some embodiments, the triggering event includes a facial recognition system detecting an identity of the patient upon the video call, entrance into the emergency department, or entrance into the ambulatory vehicle using an image sensor. Upon detection of the identity, the facial recognition system associates the patient with one or more electronic health records of the patient (from one or more electronic health record systems) using the image sensor and images stored in a database.

Upon occurrence of the triggering event 802, one or more electronic health records 804 of the patient are retrieved and data is extracted. For example, the extracted data may comprise demographics 804A, past medical history 804B, medications 804C, prior emergency visits 804D, and imaging and lab data 804E. Further, triage vitals 806 are collected for data preparation. Triage vitals 806, for example, include a pain score 806A, a temperature 806B, a blood pressure 806C, a respiratory rate 806D, an oxygen saturation level 806E, and a heart rate 806F. The one or more electronic health records 804 and the triage vitals 806 are further prepared for data processing 808.

Data processing 808 may include using natural language processing to map and bin the data to categories (e.g., chief complaints and medications) and subcategories (e.g., active ingredients in the medications having a known particular side effect). For example, an associative rule-based learning technique may be used in conjunction with an NLP technique to parse a set of textual data associated with a particular category and/or subcategory. In some embodiments, binning the categorical and sub-categorical data may include generating a series of data entries (e.g., within a database corresponding to an electronic health record system) for one or more particular categories and mapping one or more language attributes of the series subcategories based on common attributes. An associative rule-based learning technique may be used in conjunction with the NLP technique to ascertain a lexical context in which the data entries are used to identify attributes, meanings, and/or context.

Data processing 808 may also include transforming numerical features from the electronic health record and the triage vitals using a statistical imputation. For example, missing values in a column may be identified and replaced by calculating a statistical value for each column and replacing the missing value with the statistic (e.g., the mean). Values in a column may include values from one or more electronic medical records corresponding to the particular patient or a plurality of patients that have at least one similarity with respect to a category and/or subcategory (e.g., the plurality of patients and the particular patient are within the same age range and each have had at least one stroke).

Further, acuity level predictor 810 uses a machine learning algorithm to determine an acuity level for a patient based on inputting features into the acuity level predictor 810. For example, the algorithm may calculate an importance feature at each level of a decision tree. Each level may have a different threshold based on the particular features of each patient and based on the prior importance feature determination. Further, each tree in the forest may be trained with a splitting criterion ensuring division of training data such that the instances in each child node, maximally agree on a solver of preference. In embodiments, partitioning stops when an amount of data in the child node is too small. In some embodiments, each tree is trained on a random subset of 70% of the data and a random subset of 30% considered features to ensure variation and to validate findings. Further, upon presentation of a new instance, each tree may vote for the best solver based on its data.

In embodiments, the tree-based approach is suitable for classification problem(s) with multi-class classification. For example, the decision tree model works wherein the root node is the condition(feature) on which split is decided using the Gini index method. This tree-based approach is computationally fast and highly scalable. In practice, an application of the tree-based approach was compared to prior predictions by a trained human. The comparison showed that the tree-based approach was 89% accurate compared to the 68% accuracy of the predictions by the trained human. As such, turning to accuracy and verification 812, the accuracy of the model may be calculated using a Confusion matrix and further validated using a statistical test Area under the Curve (AUC). In practice, an AUC of 0.91 was achieved with using the full list of variables: patients' age, gender, arrival time, arrival mode, triage vital signs, chief complaint, prior hospital, and emergency department admissions, number of procedures and surgeries listed in the patient's record, medical history, medications, and number of orders for imaging. For example, an example calculation of the metrics required to measure algorithm performance is reproduced below:

TABLE 2
AUC Calculation

| | | |
|---|---|---|
| AUC | 0.864733 | |
| Cutoff | 0.343 | Optimal |
| Train_test split | 70:30:00 | Random method |

| | precision | recall | f1-score | support |
|---|---|---|---|---|
| 0 | 0.87 | 0.86 | 0.87 | 7526 |
| 1 | 0.86 | 0.86 | 0.86 | 7474 |
| accuracy | | | 0.86 | 15000 |
| macro avg | 0.86 | 0.86 | 0.86 | 15000 |
| weighted avg | 0.86 | 0.86 | 0.86 | 15000 |

Lastly, output 814 includes outputting an acuity level upon inputting features into the acuity level predictor 810. Output may include an alert or a value. The output may be transmitted to a clinician device. In some embodiments, an alert is transmitted to a device of an emergency contact. In some embodiments, the output includes an indication that the patient is emergent. In some embodiments, the output includes an indication that there is availability for a resource for a patient who was determined to have an acuity level of 3-5. In some embodiments, the alert indicates that a resource is unavailable or close to being unavailable. For example, the indication and/or alert may include a notification that beds are almost unavailable.

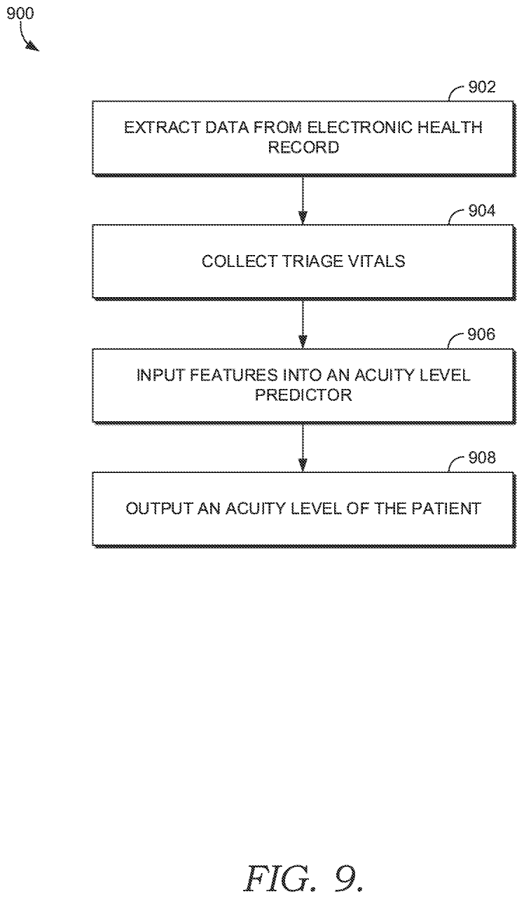

Turning to FIG. 9, FIG. 9 is a flowchart 900 for intelligently and accurately triaging a patient via an automated process. At 902, a computing system extracts data from one or more electronic health records that correspond to one or more electronic health systems upon detection of an event (e.g., a triggering event, described above in FIG. 8). The electronic health record may comprise a demographic, past medical history information, active medications, previous emergency department visits, and imaging data. The electronic health record may comprise numerical values and free-text.

At 904, the computing system collects triage vitals upon detection of the event. The triage vitals may comprise a pain score, a temperature, a respiratory rate, an oxygen saturation level, a heart rate, and a blood pressure. The triage vitals may comprise numerical values and free-text. The triage vitals may be collected automatically or by receiving input of the triage vitals. In embodiments, collecting the triage vitals includes a combination of received and automatically collected (e.g., via a temperature sensor) triage vitals.

At 906, features from the triage vitals and the data from the electronic health record are input into an acuity level predictor. Prior to input, free-text from the data and the triage vitals are mapped and binned into categorical features using natural language processing. The categorical features may include a gender, a mode of arrival, a level of consciousness, and a reason for visit. In some embodiments, the acuity level predictor uses a decision tree and a Gini index. For example, a feature importance for one or more categories is determined at each level of the decision tree. Continuing the example, each feature importance is determined using the Gini index. In some embodiments, the acuity level predictor uses a machine learning algorithm for recalculating thresholds at each level of the decision tree, and each level corresponds to one of the categorical features.

At 908, an acuity level is output by the acuity level predictor based on the input. In some embodiments, a first availability level of radiology imaging and a second availability level of hospital beds is determined for input into the acuity level predictor. In some embodiments, a probability that the patient will need a radiology service, an intensive care unit admission, and an inpatient hospitalization is determined. In some embodiments, an accuracy of the acuity level of the patient is determined using a Confusion matrix. Continuing the example, an accuracy of the probability that the patient will need the radiology service, the intensive care unit admission, and the inpatient hospitalization is determined using the Confusion matrix. Further, in some embodiments, these accuracies are validated using a statistical area under the curve. In some embodiments, a selection accepting the determined acuity level is received.

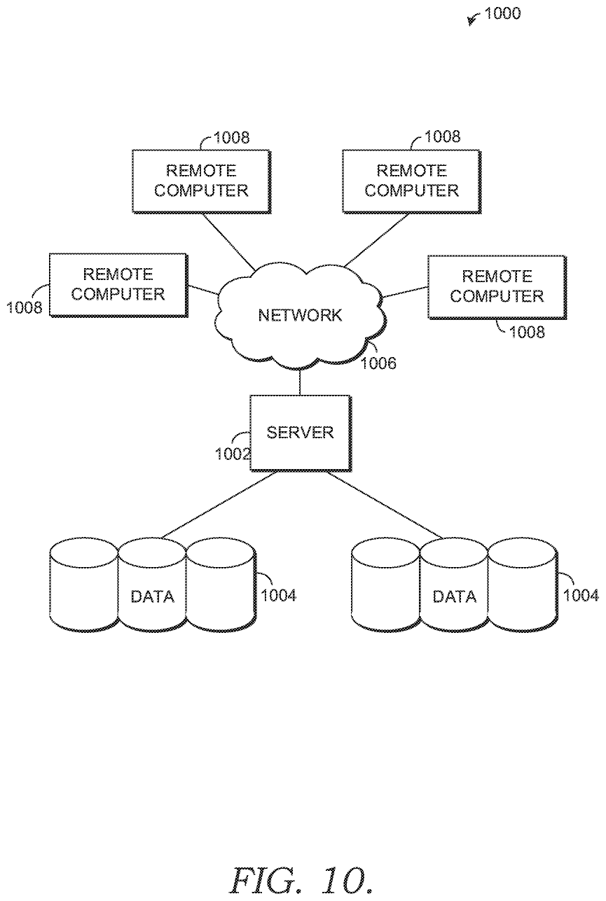

Turning now to FIG. 10, example computing environment 1000 is suitable for use in implementing embodiments of the present invention. Example computing environment 1000 is merely an example of one suitable computing environment and is not intended to suggest any limitation as to the scope of use or functionality of the invention. Neither should the computing environment 1000 be interpreted as having any dependency or requirement relating to any single component or combination of components illustrated therein.

The present invention may be operational with numerous other computing system environments or configurations. Examples of computing environments or configurations may include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

Embodiments of the present invention may be described in the general context of computer-executable instructions, such as program circuitry, being executed by a computer. Example circuitry (e.g., program circuitry or any other suitable circuitry) comprise circuits (e.g., circuits that include resistors, transistors, capacitors, inductors, diodes, and/or any suitable component whether hardware and/or programmatic code), routines, programs, objects, components, and/or data structures that perform particular tasks or implement particular abstract data types. The present invention may be practiced in distributed computing environments where tasks are performed by remote processing devices that are linked through a communications network 1006. In a distributed computing environment, program circuitry might be located in association with local and/or remote computer storage media (e.g., memory storage devices).

With continued reference to FIG. 10, the computing environment 1000 comprises a computing device in the form of a control server 1002. Exemplary components of the control server 1002 comprise a processing unit, internal system memory, and a suitable system bus for coupling various system components, including data store 1004, with the control server 1002. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 1002 typically includes therein, or has access to, a variety of computer-readable media. Computer-readable media can be any available media that may be accessed by control server 1002, and includes volatile and nonvolatile media, as well as, removable and nonremovable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program circuitry or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 1002.

Communication media typically embodies computer-readable instructions, data structures, program circuitry or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Combinations of any of the above should also be included within the scope of computer-readable media.

The control server 1002 might operate in a computer network 1006 using logical connections to one or more remote computers 1008. Remote computers 1008 might be located at a variety of locations in a medical or research environment, including clinical laboratories (e.g., molecular diagnostic laboratories), hospitals and other inpatient settings, ambulatory settings, medical billing and financial offices, hospital administration settings, home healthcare environments, clinicians' offices, Center for Disease Control, Centers for Medicare & Medicaid Services, World Health Organization, any governing body either foreign or domestic, Health Information Exchange, and any healthcare/government regulatory bodies not otherwise mentioned. Clinicians may comprise a treating physician or physicians, specialists such as intensivists, surgeons, radiologists, cardiologists, and oncologists, emergency medical technicians, physicians' assistants, nurse practitioners, nurses, nurses' aides, pharmacists, dieticians, microbiologists, laboratory experts, laboratory technologists, genetic counselors, researchers, students, and the like.

The remote computers 1008 might also be physically located in nontraditional medical care environments so that the entire healthcare community might be capable of integration on the network. The remote computers 1008 might be personal computers, servers, routers, network PCs, peer devices, other common network nodes, or the like and might comprise some or all of the elements described above in relation to the control server 1002. The devices can be personal digital assistants or other like devices.

Computer networks 1006 comprise local area networks (LANs) and/or wide area networks (WANs). Such networking environments are commonplace in offices, enterprise-wide computer networks, intranets, and the Internet. When utilized in a WAN networking environment, the control server 1002 might comprise a modem or other means for establishing communications over the WAN, such as the Internet. In a networking environment, program circuitry or portions thereof might be stored in association with the control server 1002, the data store 1004, or any of the remote computers 1008. For example, various application programs may reside on the memory associated with any one or more of the remote computers 1008. It will be appreciated by those of ordinary skill in the art that the network connections shown are exemplary and other means of establishing a communications link between the computers (e.g., control server 1002 and remote computers 1008) might be utilized.

In operation, an organization might enter commands and information into the control server 1002 or convey the commands and information to the control server 1002 via one or more of the remote computers 1008 through input devices, such as a keyboard, a pointing device (commonly referred to as a mouse), a trackball, a touch display, or a touch pad. Other input devices include microphones, satellite dishes, scanners, or the like. Commands and information might also be sent directly from a remote healthcare device to the control server 1002. In addition to a monitor, the control server 1002 and/or remote computers 1008 might comprise other peripheral output devices, such as speakers and a printer.

From the foregoing, it will be seen that this technology is one well adapted to attain all the ends and objects described above, including other advantages that are obvious or inherent to the structure. It will be understood that certain features and subcombinations are of utility and may be employed without reference to other features and subcombinations. This is contemplated by and is within the scope of the claims. Since many possible embodiments of the described technology may be made without departing from the scope, it is to be understood that all matter described herein or illustrated the accompanying drawings is to be interpreted as illustrative and not in a limiting sense.

What is claimed is:

1. A computerized method for intelligently and accurately triaging a patient, the method comprising:
accessing an electronic health record of the patient based on a detection of an event;
collecting triage vitals of the patient upon the detection of the event;
mapping and binning free-text from the electronic health record and the triage vitals into categorical features using natural language processing;
transforming numerical features from the electronic health record and the triage vitals using a statistical imputation;
in response to mapping, binning, and transforming, inputting the categorical features and the numerical features into an acuity level predictor; and
based on inputting the categorical features and the numerical features into the acuity level predictor, outputting an acuity level of the patient for triaging the patient.

2. The method of claim 1, wherein the acuity level of the patient comprises one of the following: resuscitation, emergent, urgent, less urgent, and non-urgent.

3. The method of claim 1, wherein the electronic health record comprises a demographic, past medical history information, active medications, previous emergency department visits, and imaging data, and wherein the electronic health record was previously stored in a database.

4. The method of claim 3, wherein the event comprises the patient entering an emergency department and a facial recognition system associating the patient with the electronic health record of the patient using an image sensor and images stored in the database.

5. The method of claim 1, wherein the triage vitals comprise a pain score, a temperature, a respiratory rate, an oxygen saturation level, a heart rate, and a blood pressure.

6. The method of claim 1, wherein the categorical features include a gender, a mode of arrival, a level of consciousness, and a reason for a visit.

7. The method of claim 6, wherein the numerical features include an age, a number of hospital visits during a period of time, and a blood pressure.

8. The method of claim 1, further comprising:
determining a first availability level of radiology imaging;
determining a second availability level of hospital beds; and
outputting the acuity level of the patient upon inputting the first availability level and the second availability level into the acuity level predictor.

9. The method of claim 8, further comprising:
determining, using the acuity level, a probability that the patient will need a radiology service, an intensive care unit admission, and an inpatient hospitalization.

10. The method of claim 1, wherein the acuity level predictor uses a decision tree and Gini index for outputting the acuity level based on inputting the categorical features and the numerical features.

11. The method of claim 1, further comprising determining an accuracy of the acuity level of the patient using a Confusion matrix.

12. The method of claim 11, further comprising validating the accuracy of the acuity level using a statistical area under the curve.

13. One or more non-transitory computer storage media storing computer-readable instructions that, when executed by a processor, cause the processor to perform operations for intelligently and accurately triaging a patient, the operations comprising:
accessing an electronic health record of the patient based on a detection of an event;
collecting triage vitals of the patient upon the detection of the event;
mapping and binning free-text from the electronic health record and the triage vitals into categorical features using natural language processing for input into an acuity level predictor;
inputting the categorical features into the acuity level predictor that uses a machine learning algorithm for recalculating thresholds at each level of a decision tree, each level corresponding to one of the categorical features; and
based on inputting the categorical features into the acuity level predictor, outputting an acuity level of the patient for triaging the patient.

14. The media of claim 13, further comprising inputting numerical features from the electronic health record and the triage vitals, the numerical features comprising a blood pressure, a pain score, a heart rate, a temperature, a number of procedures performed by a clinician during a time range, and an age.

15. The media of claim 13, wherein the acuity level of the patient comprises one of the following: resuscitation, emergent, urgent, less urgent, and non-urgent.

16. The media of claim 15, further comprising determining a probability that the patient will need a radiology service, an intensive care unit admission, and an inpatient hospitalization based on the acuity level.

17. The media of claim 16, further comprising determining an accuracy of the acuity level, the probability that the patient will need the radiology service, the probability that the patient will need the intensive care unit admission, and the probability that the patient will need the inpatient hospitalization using a Confusion matrix.

18. The method of claim 17, further comprising validating the accuracy of the acuity level, the probability that the patient will need the radiology service, the probability that the patient will need the intensive care unit admission, and the probability that the patient will need the inpatient hospitalization using a statistical area under the curve.

19. An intelligent triage system for triaging a patient, the system comprising:
at least one processor; and
one or more computer storage media storing computer-executable instructions embodied thereon that when executed by the at least on processor, cause the at least one processor to perform operations comprising:
retrieve an electronic health record of the patient based on a detection of an event;
collect triage vitals of the patient upon the detection of the event;
map and bin free-text from the electronic health record and the triage vitals into categorical features using natural language processing for input into an acuity level predictor;
input the categorical features into the acuity level predictor that recalculates a threshold at each level of a decision tree, each level corresponding to one of the categorical features; and
based on inputting the categorical features into the acuity level predictor, output an acuity level of the patient for triaging the patient.

20. The system of claim 19, further comprising receiving a selection accepting the acuity level.
